(19) United States
(12) Patent Application Publication
(10) Pub. No.: US 2004/0018261 A1
(43) Pub. Date: Jan. 29, 2004

(54) Method and use of extract of a member of Typhaceae's family

(76) Inventor: Tong-Shui Zhou, Shanghai (CN)

Correspondence Address: Raymond Y. Chan, Suite 128, 108 N. Ynez Ave., Monterey Park, CA, 91754, US

(21) Appl. No.: 10/621,426

(22) Filed: Jul. 16, 2003

(30) Foreign Application Priority Data
Jul. 19, 2002 (CN) 02136115.0

(51) Int. Cl. A61K 35/78; A61K 31/7048

(52) U.S. Cl. 424/778; 514/27

(57) ABSTRACT

An extract of Typhae Pollen which is a traditional Chinese medicine consisting of flavonoids as active components includes at least one member of the group selected from kaempferol, quercetin and isorhamnetin and the derivatives of these active components. The present invention also relates to the degraded form of flavonoids, the metal derivatives formed with sodium and potassium salts, and the metal complex formed with a predetermined metal ion. The extract of Typhae Pollen is prepared by a plurality of extraction processes and has a plurality of functions for promoting health including lowering blood lipid level, preventing arteriosclerosis, promoting tolerance of brain and heart tissue under anaerobic condition, preventing blood platelet coagulation, preventing thrombosis and stop bleeding. The extract of Typhae Pollen of the present invention is also used for preventing and treating diseases related to blood vessels in brain and heart, and poor blood circulation induced diseases selecting from the group consisting of chest pain, stomachache, physical injuries, puerperium pain and menstruation.

### BACKGROUND OF THE PRESENT INVENTION

1. Field of Invention

The present invention relates to technology in medical and health science, and more particularly to an extract of a Typhae Pollen and its manufacture and application in medicine and health science.

2. Description of Related Arts

‘Typhae Pollen’ is a common traditional Chinese medicine, which is generally dried pollen of the family Typhaceae such as Typha angustifolia L. and Typha orientalis Presl. According to the principles of the tradition Chinese medicine, Typhae Pollen has the properties of stop bleeding, bruise heeling, and enhancing circulation of the lymphatic system and it has been widely used in the treatment of bleeding, apistaxis, hematemesis, external bleeding, painful menstruation, colic, abscess, painful lymphatic system's disease or discomfort. Recent scientific researches envisage that the extracted components of Typhae Pollen by water extraction or alcohol extraction is capable of substantially increasing the coronary blood flow, improving microcirculation, increasing the tolerance ability of brain and cardiac muscle under anaerobic condition, lowering the consumption of oxygen of brain and heart system, promoting blood vessel dilation, lowering the blood lipid level, preventing arteriosclerosis, and acting as anticoagulant. All the different species of Typhae Pollen comprises organic acid, flavonoids, sterol components, long chain aliphatic components and polysaccharides. The principles and applications of these chemical components were only once disclosed in a Chinese patent 1006015 in China wherein the active mechanism and application of lowering blood lipid level of sterol, long chain aliphatic compounds of Typhae Pollen were described. Since then, there is no related arts relating to Typhae Pollen's extract.

### SUMMARY OF THE PRESENT INVENTION

A main object of the present invention is to provide an extract of Typhae Pollen and a manufacturing method thereof wherein one of the extract components is flavonol glycosides.

Another object of the present invention is to provide an extract of Typhae Pollen wherein one of the extracted components is a degraded form of flavonol glycosides.

Another object of the present invention is to provide an extract of Typhae Pollen wherein one of the extract components, namely flavonol glycosides, reacts with a predetermined alkali or metal salt selected from a predetermined group to form a predetermined derivative.

Another object of the present invention is to provide an extract of Typhae Pollen wherein one of the extract components, namely flavonol glycosides, reacts with a predetermined metal ion selected from a predetermined group to form a predetermined metal complex.

Another object of the present invention is to provide an application of an extract of Typhae Pollen, including a degraded form, a derivative and a metal complex of the predetermined extract of Typhae Pollen.

Another object of the present invention is to provide an extract of Typhae Pollen wherein one of the extract components is flavonol glycosides and the active components of the flavonol glycosides are a combination of the group selected from general structural formulae (A), (B) and (C). The general structural formula (A) consists of chemical components (1), (2), (3) and (4); the general structural formula (B) consists of chemical components (5), (6), (7) and (8); and the general structural formula (C) consists of chemical components (9), (10), (11), (12) and (13). The general structural formula (A) is:

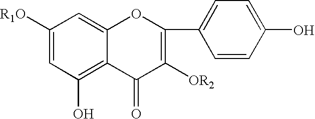

where

(1) R₁=R₂=H;

(2) R₁=H, R₂=neohesperidoside;

(3) R₁=H, R₂=(2^{G}-rham)-rutinoside

(4) R₁=rhamnoside, R₂=rutinoside

The general structural formula (B) is:

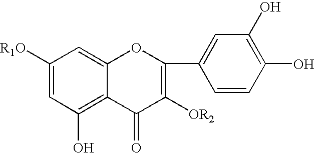

where

(5) R₁=R₂=H;

(6) R₁=H, R₂=neohesperidoside;

(7) R₁=H, R₂=(2^{G}-rham)-rutinoside

(8) R₁=rhamnoside, R₂=rutinoside

The general structural formula (C) is:

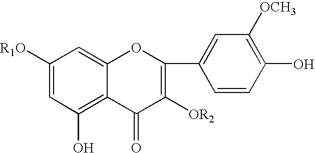

where

(9) R₁=R₂═H;

(10) R₁=H, R₂=neohesperidoside;

(11) R₁=R₂=rutinoside

(12) R₁=H, R₂=(2^{G}-rham)-rutinoside

(13) R₁=rhamnoside, R₂=rutinoside

The chemical components (1), (2), (3), (4), (5), (6), (7), (8), (9), (10), (11), (12) and (13) are kaempferol, kaempferol-3-O-neohesperidoside, kaempferol-3-O-(2^{G}-α-L-rham)-rutinoside, kaempferol-3-rutino-7-rhamnoside, quercetin, quercetin-3-O-neohesperidoside, quercetin-3-O-(2^{G}-α-L-rham)-rutinoside, quercetin-3-rutino-7-rhamnoside, isorhamnetin, isorhamnetin-3-O-neohesperidoside, isorhamnetin-3-O-(2^{G}-α-L-rham)-rutinoside and isorhamnetin-3-rutino-7-rhamnoside respectively.

Accordingly, in order to accomplish the above objects, the present invention provides an extract of Typhae Pollen consisting of flavonoids as active components comprising at least one member of the group selected from kaempferol, kaempferol-3-O-neohesperidoside, kaempferol-3-O-(2^{G}-α-L-rham)-rutinoside, kaempferol-3-rutino-7-rhamnoside, quercetin, quercetin-3-O-neohesperidoside, quercetin-3-O-(2^{G}-α-L-rham)-rutinoside, quercetin-3-rutino-7-rhamnoside, isorhamnetin, isorhamnetin-3-O-neohesperidoside, isorhamnetin-3-O-(2^{G}-α-L-rham)-rutinoside and isorhamnetin-3-rutino-7-rhamnoside.

These and other objectives, features, and advantages of the present invention will become apparent from the following detailed description, the accompanying drawings, and the appended claims.

### DETAILED DESCRIPTION OF THE PREFERRED EMBODIMENT

The Typhae Pollen of the present invention is a plant belonging to the family Typhaceae. Typhae Pollen comes from any plants belonging to any members of the family Typhaceae. The scientific definition and genus classification of Typhaceae may be referred to the “China Higher Plants Classification Dictionary”, revised edition, P. 506 (Editor: Hou Kuan Zhao, Beijing Science Publisher, 1984.12). The crude materials of Typhae Pollen may be chosen from the pollen, spica, fruit, stem, leave, underground stem, root, any portions of the plant or the whole plant of Typhaceae wherein the preferred crude materials is the mature pollen of the flower of the plant belonging to Typhaceae. The Typhae Pollen of the present invention does not only include the raw pollen of the flower of the plant which is not treated, namely common “Raw Typhae Pollen”, but also includes any semi-products or treated pollen of the flower of the plant such as “Fried Typhae Pollen”, “Burnt Typhae Pollen”, “Alcoholic Typhae Pollen” and “Vinegar Typhae Pollen”.

The extract of Typhaceae of the present invention includes the extract obtained from any portions of any members of the plant belonging to the family Typhaceae which contains a combination of active components wherein the first preferred embodiment is extracted and manufactured from the mature pollen of any members of Typhaceae which contains a variety of combination of active components. The active components include flavonoids such as kaempferol, quercetin, and isorhamnetin and their derivatives.

The extract of Typhaceae of the present invention has a total percentage composition of different kinds of flavonoids between 5% and 100% (w/w), whereas a preferred total percentage composition is between 50% and 100% (w/w) and a first preferred total percentage composition is between 95% and 100% (w/w).

The extract of Typhaceae of the present invention also includes degraded forms of flavonol glycosides prepared from heating under a predetermined long time, partial degradation under acidic or alkaline conditions, or enzyme reaction. The degraded or partially degraded forms of flavonol glycosides include 3-O-glycoside, 3-O-rutinoside, and 7-O-rhamnoside of flavonoids such as isorhamnetin, kaempferol, and quercetin. The heating under a predetermined long time means heating under 40-100° C. for more than one hour. The acid used may be inorganic acid such as hydrochloric acid, sulfuric acid, phosphoric acid, nitric acid, nitrous acid, sulfurous acid, carbonic acid (or aqueous carbon dioxide solution) and hydrofluoric acid. The acid may also be organic acid such as methane acid, ethanoic acid, glacial acetic acid, trichloroacetic acid, acetic anhydride, citric acid and ethanedioic acid. The alkali used may be potassium hydroxide, sodium hydroxide, sodium hydrogen carbonate, sodium bicarbonate, pyridine, ammonia water, diethylamine and triethylamine. The enzyme used may be selected from different kinds of glycoside bonding hydrolytic enzymes such as invertase, maltase, Armeniacae Amarum Semen enzyme, cellulase, Hodmandod's enzyme, hesperidosidase, and citrin. The above acid, alkaline and enzyme degradation reactions are normally carried out under a predetermined heating condition.

The extract of Typhaceae of the present invention is capable of forming a predetermined metal salt derivative by reaction with a predetermined alkali or metallic salt such as sodium salt or potassium salt including potassium hydroxide, sodium hydroxide, sodium hydrogen carbonate, sodium bicarbonate and sodium acetate. The derivatives generally have the same principles and applications as the extract of Typhaceae of the present invention. Mixing the extract of Typhaceae of the present invention with a 0.01-5N alkali solution or a predetermined salt in solution and heating to keep a predetermined temperature will result in the production of the predetermined derivative.

The extract of Typhaceae of the present invention is capable of forming a predetermined metallic complex salt by reaction with a predetermined metal ion such as iron, zinc, magnesium, chromium, aluminum, copper, calcium, cobalt, barium, strontium, and zirconium ions. The metallic complex salts generally do not only have the same principles and applications as the extract of Typhaceae of the present invention, but also may have some new additional applications. For examples, an iron complex is capable of being used as iron supplement; a zinc complex is capable of being used as a zinc supplement; and a chromium complex is capable of being used to prevent diabetes. In a water or acetate solution of the extract of Typhaceae of the present invention, a 0.01-5N metallic complex salt is added and mixed under a predetermined desirable temperature which gives rise to the formation of the predetermined metallic complex.

According to the present invention, the active components of the extract of Typhaceae are different kinds of flavonoids wherein the two major active components are isorhamnetin-3-O-(2^{G}-α-L-rham)-rutinoside and isorhamnetin-3-O-neohesperidoside, wherein the isorhamnetin-3-O-(2^{G}-α-L-rham)-rutinoside and/or isorhamnetin-3-O-neohesperidoside have a percentage composition between 20% to 100% (w/w) and a preferred percentage composition of the two major active components is between 50% and 100% (w/w) while a first preferred percentage is between 95% and 100% (w/w).

The extract of Typhaceae of the present invention is prepared by a preparation process or a combination of preparation processes which may be: (1) solvent extraction; (2) macroporous resin adsorption; (3) lead precipitation; (4) supercritical CO₂ extraction; (5) column chromatography; and/or (6) Liquid-Liquid reflux extraction, where a preferred process for preparing the extract of Typhaceae of the present invention is (2) macroporous resin adsorption and/or (5) column chromatography.

The above preparation process generally includes techniques and procedures of (a) extraction; (b) filtration; (c) condensation and (d) drying.

(a) Extraction: a predetermined solvent for extraction may be a predetermined solution of water, acetate, ketone or ester, a mixture having a predetermined concentration of the solution, or a predetermined acidic or alkaline solvent prepared from reacting the predetermined solvent for extraction with acid or alkaline where a preferred solvent is 70% ethanol. Applicable extraction methods are refluxation, diffusible extraction, ultraextraction, microextraction and high pressure extraction.

(b) Filtration: filtration methods include centrifugation, extract filtration, pressure filtration, and ultrafiltration with or without the use of one of the following fining agents or a combination of the fining agents: gelatin, active charcoal, infusorial earth, china clay; resins, polyethylene glycol, polyethene triol, chitsoan, and natural fining agents such as ‘101 juice fining agent’ and ‘ZTC+1 natural fining agent’ available in the market.

(c) Condensation: Methods of condensation include normal ambient pressure or reduced pressure condition's film evaporation, rotatory evaporation and continuous evaporation by heating.

(d) Drying: Methods of drying include vacuum drying, spray drying and freeze drying.

When the solvent extraction is used in the preparation process, a predetermined extract of Typhaceae of the present invention is first soaked with water. Then a predetermined solvent selecting from the group of a low polarity ether group, alkane and ester solvent (such as a petroleum ether, ethylether, hexane, ethyl acetate, or gasoline) is used to extract oil soluble impurities from the extract of Typhaceae of the present invention. Lastly, a predetermined extraction solvent having a predetermined desirable polarity, such as butanol, isopropanol, chloroform, or a combination of the extraction solvents is used to extract out a flavonoid extract constituent wherein a first preferred solvent is butanol.

When the macroporous resin adsorption is used in the preparation process, a predetermined resin such as a non-polar, a low polarity, a medium polarity, a low basicity, or a low acidity type resin is used. The resin available in the market comes from different manufacturers of different countries, for examples, D101 from Tian Jin Agricultural Medicine Factory (a PRC company), DA20, HZ-802, HZ-806, 1300, 1400 from Shanghai Hua Zhen Technology Trading Company, 860021, and DM130 from Shan Dong Lu Kang Medical Group (a PRC company). A preferred resin is a non-polar adsorption resin such as D101, HZ-802 and DM130. A predetermined extracting agent is used which may be water, or ethanol, methanol or propanone in water, wherein a preferred extracting agent is 0-100% ethanol.

When the Lead salt precipitation is used in the preparation process, a predetermined lead salt agent is used which may be lead acetate or basic lead acetate where a predetermined demineralizing agent is H₂S, phosphate or sulfate.

When the supercritical CO₂ extraction is used in the preparation process, a flavonoid extract is capable of directly extracted from crude materials of Typhaceae, or extracted from the extract of Typhaceae of the present invention prepared from the preparation processes: (1) solvent extraction, (2) macroporous resin adsorption or (3) lead precipitation. A predetermined solvent for supercritical CO₂ extraction or a combination may or may not be used in the supercritical CO₂ extraction wherein the solvent used may be water, acetate, ketone or ester.

When the column chromatography is used in the preparation process, starting reactants used in the column chromatography are the extract of Typhaceae produced from the above preparation processes: (1) solvent extraction; (2) macroporous resin adsorption; (3) lead precipitation; and (4) supercritical CO₂ extraction; or the starting reactants are preliminary refining products produced from (1) solvent extraction; (2) macroporous resin adsorption; (3) lead precipitation or (4) supercritical CO₂ extraction. A predetermined stationary bed is used in the column chromatography which is selected from the group consisting of silica gel, polyamide, aluminum oxide, polysaccharide (Sephadex series or Sephadex—LH20 series), C-8, C-18, active charcoals, and cellulose. A predetermined eluting agent is used with respect to the predetermined stationary bed and is generally selected from the group consisting of water, methanol, ethanol, propanone, chloroform, ethyl acetate or their mixtures, or is a mixture of the group wherein a preferred stationary bed is the silica gel or the Sephadex series polysaccharide.

When the Liquid-Liquid reflux extraction is used in the preparation process, starting reactants used are predetermined products produced from the above preparation processes: (1) solvent extraction; (2) macroporous resin adsorption; (3) lead precipitation; (4) supercritical CO₂ extraction and (5) column chromatography, or are preliminary refining products produced from (1) solvent extraction; (2) macroporous resin adsorption; (3) lead precipitation; (4) supercritical CO₂ extraction and (5) column chromatography. A predetermined flavonoid extract is first mixed with water and oil soluble impurities are removed with the use of low polarity ester, alkane or ether solution such as petroleum ether, ethylether, hexane, ethyl acetate and gasoline. Then a predetermined solvent having a predetermined polarity selected from the group consisting of butanol, isopropanol and chloroform or a mixture of the predetermined solvent is used for extracting a flavonol glycosides constituent wherein a first preferred extracting agent is butanol.

Animal tests have been conducted for testing the effects of the extract of Typhaceae of the present invention so as to envisage its obvious functions on coronary vessels' diseases and bleeding. The experiments and results are as follows:

Experiment 1: Acute Toxin Test

A predetermined number of rats are provided and each rat is feed with a predetermined quantity of the extract of Typhaceae of the present invention. An acute lethal dose level of the rats is calculated as LD50(g/kg) and is 3.285 g/kg by BLISS calculation.

Experiment 2: The Effect of Lowering Blood Lipid Level and Preventing Arteriosclerosis

Rabbits of either sex having body weight of 2.0+0.2 kg are selected and divided into 5 groups, namely a control group; a light dose group; a heavy dose group; a sodium derivative group; and an aluminum complex group. Each group consists of 10 rabbits and each rabbit is feed by 0.2 g/kg cholesterol (dissolved in 2 ml lard oil) per day per feed. The light dose group is feed with a 10 mg/kg extract of Typhaceae of the present invention. The heavy dose is feed with a 30 mg/kg extract of Typha of the present invention. The sodium derivative and the aluminum complex groups are feed with a 30 mg/kg sodium derivative extract and a 30 mg/kg aluminum complex extract of the present invention respectively. The control group is feed with a physiological saline solution two times per day for 8 weeks wherein the volume of physiological saline solution being feed is equal to the size of the rabbits. At the 2^{nd}, 4^{th}, 6^{th}, and 8^{th} week, venal blood is extracted from the ear of each rabbit and is centrifuged at 300 rpm to obtain a serum sample.

The method of testing total cholesterol in the serum sample and triglyceride testing can be found in the reference for experiment 2 ‘Research Methods of the Principles of Chinese Medicine’ (Editor: Chen Qi, Beijing R1 Min Wei San Publisher, 1993, P. 630 and P. 624). Results are summarized in table 1 and table 2 as follows:

TABLE 1
The effect of the extract of Typhaceae of the present invention on total cholesterol level

| | Number | | Total Cholesterol level (mg/kg) (±SD) | | | | |
|---|---|---|---|---|---|---|---|
| | of | Extract | Normal | | | | |
| Group | animals | (mg/kg) | value | Week 2 | Week 4 | Week 6 | Week 8 |
| Control | 10 | physiological | 80.20 ± 10.08 | 376.35 ± 21.40 | 560.45 ± 70.80 | 590.54 ± 0.31 | 694.35 ± 50.80 |
| | | saline solution | | | | | |
| Light dose | 10 | 10 | 82.15 ± 15.54 | 330.63 ± 53.36 | 320.80 ± 68.75** | 310.58 ± 34.19** | 293.51 ± 20.30*** |
| Heavy dose | 10 | 30 | 78.30 ± 11.58 | 299.16 ± 30.16 | 263.56 ± 7.30** | 230.29 ± 18.65*** | 160.34 ± 23.60*** |
| Sodium | 10 | 30 | 77.20 ± 10.70 | 296.50 ± 9.08 | 260.30 ± 8.96** | 224.93 ± 13.80*** | 158.26 ± 10.08*** |
| derivative | | | | | | | |
| Aluminum | 10 | 30 | 79.42 ± 12.76 | 300.64 ± 9.08 | 265.43 ± 11.64** | 232.39 ± 12.44*** | 162.65 ± 12.83*** |
| complex | | | | | | | |

**P < 0.05,
***P < 0.01

TABLE 2
The effect of the extract of Typhaceae of the present invention on serum's triglyceride

| | Number | | | | | | |
|---|---|---|---|---|---|---|---|
| | of | Extract | Triglyceride level (mg/kg) (±SD) | | | | |
| Group | animal | (mg/kg) | Normal value | Week 2 | Week 4 | Week 6 | Week 8 |
| Control | 10 | physiological | 12.78 ± 2.45 | 14.80 ± 1.80 | 22.78 ± 3.56 | 34.20 ± 6.50 | 58.60 ± 7.85 |
| | | saline | | | | | |
| | | solution | | | | | |
| Light dose | 10 | 10 | 13.89 ± 1.80 | 14.75 ± 3.60 | 20.44 ± 5.85 | 21.35 ± 4.62** | 22.45 ± 4.60*** |
| Heavy | 10 | 30 | 14.58 ± 1.65 | 13.98 ± 2.56 | 13.86 ± 4.35 | 12.89 ± 2.56*** | 11.86 ± 1.75*** |
| dose | | | | | | | |
| Sodium | 10 | 30 | 13.64 ± 1.78 | 13.44 ± 1.89 | 13.06 ± 3.23 | 12.22 ± 1.33*** | 10.68 ± 2.56*** |
| derivative | | | | | | | |
| Aluminum | 10 | 30 | 13.96 ± 2.43 | 13.88 ± 1.35 | 13.67 ± 3.32 | 13.16 ± 2.05*** | 12.33 ± 2.66*** |
| complex | | | | | | | |

**P < 0.05,
***P < 0.01

After 8 weeks, the rabbits are killed and their aorta (portion from heart to iliac artery) are collected and that the lipid tissue outside the aorta are removed. Then the aorta are cut along the rear side longitudinally and displayed in a container, where the aorta are soaked with 0.5 ml/ml formaldehyde solution for 24 hours and stained by sudan III solution for 30 minutes to form stained aorta samples. The stained aorta samples are colored as golden red from ivory patch. According to the reference for experiment 2 (see above, P. 626), the aorta samples are graded and the patch area of the aorta samples are analyzed so as to obtain a patch index and a tolerance percentage. Results are summarized in table 3.

TABLE 3
The effect of the extract of Typhaceae of the present invention on
arteriosclerosis of rabbits

| | | | Patch Index | Tolerance |
|---|---|---|---|---|
| | Number of | | (±SD) Normal | Percentage |
| Group | animal | Extract (mg/kg) | value | (%) |
| Control | 10 | Physiological | 3.05 ± 0.32 | — |
| | | saline solution | | |
| Light dose | 10 | 10 | 1.76 ± 0.05*** | 49.7 |
| Heavy dose | 10 | 30 | 0.80 ± 0.03*** | 77.1 |
| Sodium | 10 | 30 | 0.78 ± 0.12*** | 79.6 |
| derivative | | | | |
| Aluminum | 10 | 30 | 0.82 ± 0.23*** | 76.3 |
| complex | | | | |

***P < 0.01

Experiment 3: The Effect on Dissected Coronary Artery Blood Flow and Heartbeat Rate of Rabbit

The extract of Typhaceae of the present invention is made into a 1 mg/ml extract solution for experiment 3, which is purified by centrifugation and that its pH is set as 7.0. The sodium derivative and the aluminum complex of the present invention are also prepared into a 1 mg/ml sodium derivative and a 1 mg/ml aluminum solution for experiment 3. A ‘Salvia Miltiorrhiza solution’ (each is a 2 ml solution, each ml is equal to 1 g Salvia Miltiorrhiza and 1 g ‘Xiang Xiang’, a product manufactured by Shanghai Number 9 Pharmaceutical Company in PRC) is used as control solution.

Experimental groups are divided into a low dose group (0.5 ml), a high dose group (1.5 ml), a sodium derivative group (1.5 ml), an aluminum complex group (1.5 ml) and a control group (1.5 ml). Each group consists of 10 rabbits. Each dissected heart of each rabbit is prepared according to Langendoff method, which is then connected to a perfusion instrument. After a steady heartbeat rate is obtained, a coronary blood flow is analyzed for 30 seconds and a heartbeat chart is scanned simultaneously for obtaining a pre-test data. Then, each rabbit is injected with a predetermined testing solution according to the experimental groups, namely a 0.5 mg/ml extract solution, a 1.5 mg/ml extract solution, a 1.5 ml sodium derivative solution, a 1.5 ml aluminum complex solution and a 1.5 ml control solution, from the anterior portion of the dissected heart. After 5 seconds, coronary blood flow is collected for 30 seconds and amplitude of heartbeat chart is recorded simultaneously for obtaining a resulting data. The pre-test data and the resulting date are compared and shown in table 4 and table 5.

TABLE 4
The effect of the present invention on coronary blood flow of rabbits

| | Number | Testing | | |
|---|---|---|---|---|
| | of | solution | Coronary flow rate (±SD, ml/30 s) | |
| Group | animal | (ml) | Before experiment | After experiment |
| Light dose | 10 | 0.5 | 6.84 ± 1.25 | 9.85 ± 1.86** |
| Heavy dose | 10 | 1.5 | 6.73 ± 1.04 | 17.02 ± 2.18*** |
| Sodium | 10 | 1.5 | 6.98 ± 1.04 | 18.52 ± 1.88*** |
| derivative | | | | |
| Aluminum | 10 | 1.5 | 7.12 ± 1.04 | 16.35 ± 2.43*** |
| complex | | | | |
| Control | 10 | 1.5 | 7.08 ± 1.12 | 15.18 ± 1.68*** |

**P < 0.05,
***P < 0.01

TABLE 5
The effect of the present invention on contraction amplitude
of heart of rabbits

| | | | Contraction amplitude | |
|---|---|---|---|---|
| | | | (±SD, ml/30 s) | |
| | Number of | Testing | Before | After |
| Group | animal | solution (ml) | experiment | experiment |
| Light dose | 10 | 0.5 | 2.78 ± 0.58 | 4.12 ± 0.64** |
| Heavy dose | 10 | 1.5 | 2.84 ± 0.65 | 4.68 ± 0.88*** |
| Sodium | 10 | 1.5 | 2.66 ± 0.65 | 5.02 ± 0.06*** |
| derivative | | | | |
| Aluminum | 10 | 1.5 | 2.92 ± 0.65 | 4.15 ± 0.56*** |
| complex | | | | |
| Control | 10 | 1.5 | 2.86 ± 0.51 | 3.78 ± 0.74* |

*P > 0.05,
**P < 0.05,
***P < 0.01

Experiment 4: The Effect of Post Pituitary Hormone on Acute Cardiac Blood Deficiency of Rats

The testing solutions used in experiment 4 are the same as the testing solutions in experiment 3.

Forty-eight Wistar rats are provided where male to female ratio is 1:1 and are randomly divided into 6 groups. Inject 0.5 ml/kg post pituitary hormone through femoral vein and scan at 15-second, 30-second, 45-second, 1-minute, 1-minute-30-second, 2-minute-30-second, 3-minute, 4-minute, 5-minute intervals cardiogram and take away those rats without blood deficiency. Wait till the cardiogram of the rate become normal and inject the predetermined testing solution through femoral vein. After 5 minutes, inject post pituitary hormone while same weight physiological saline solution is used for control group. Cardiogram is obtained by the same method as described above and the ST increasing section and T-curve maximum percentage increase are observed and results are shown in table 6.

TABLE 6
The effect of the present invention on post pituitary hormone
of rats leading to cardiac blood deficiency cardiogram (±SD)

| | ST increasing section | T-curve maximum |
|---|---|---|
| Group | maximum tolerance (%) | percentage increase (%) |
| Physiological saline | 2.70 ± 1.50 | 1.02 ± 0.24 |
| solution | | |
| Salvia miltiorrhiza | 25.04 ± 11.75** | 32.98 ± 7.64** |
| solution | | |
| Light dose | 22.14 ± 5.89** | 26.94 ± 7.70** |
| Heavy dose | 26.12 ± 8.09** | 34.78 ± 6.85* |
| Sodium derivative | 27.33 ± 4.18** | 36.53 ± 4.065* |
| Aluminum complex | 24.78 ± 6.34** | 28.65 ± 3.35* |

n = 8,
*P > 0.05,
**P < 0.05

Experiment 5: The Effect of the Present Invention on Blood Platelet of ADP Directed Rabbits

The testing solutions used in experiment 5 are the same as the testing solutions in experiment 3.

Fifty rabbits are selected and randomly divided into 5 groups: a control (physiological saline solution) group; a light dose group; a heavy dose group; a sodium derivative group; and an aluminum complex group. Each group consists of 10 rabbits. Each rabbit is injected with a predetermined testing solution according to their group while physiological saline solution is used for control group. After 4 hours, venal blood is extracted from the ear. 3.8% sodium citrate is added where the ratio of sodium citrate to the testing solution is 1:9 for anticoagulation and, by centrifugation, nutritious blood platelet plasma (PRP, 1000 r/min, 7 min) and anemia blood platelet plasma (PPP, 4000 r/min, 10 min) are collected. Before measurement, a platelet aggregometer is set to zero and a PRP is used to set at the 10^{th} square of the record paper where the PPP is used to set at the 80^{th} square of the record paper (There is a total 100 squares on the recording paper). Transfer 200 ul PRP to a test tube and water bath at 37° C. for 5 minutes. Then add 20 ul of 5 u mol ADP and record the maximum tolerance percentage within a 5-minute period, hence calculate the aggregation tolerance rate. Aggregation tolerance rate=((maximum aggregation rate of control group-maximum aggregation rate of testing group)/maximum aggregation rate of control group)×100%. Results are shown in table 7.

TABLE 7
The effect of the extract of Typhaceae of the present invention
on blood platelet of ADP directed rabbits

| | | Blood platelet | Aggregation |
|---|---|---|---|
| | | aggregation | tolerance rate |
| Group | Quantity (ml) | rate (%) | (%) |
| Physiological saline | — | 51.5 ± 5.6 | — |
| solution | | | |
| Light dose | 1 | 28.8 ± 6.8** | 44.1 |
| Heavy dose | 3 | 17.9 ± 3.8** | 65.2 |
| Sodium derivative | 3 | 16.7 ± 2.3** | 67.6 |
| Aluminum complex | 3 | 18.5 ± 4.2** | 64.1 |

Compare with control,
**P < 0.01

Experiment 6: The Effect of the Present Invention on Electrical Stimulus Resulting Carotic Thrombosis of Rabbits

Sixty rabbits are selected and randomly divided into 6 groups: a control (physiological saline solution) group; a urokinase group, a low dose group; a heavy dose group; a sodium derivative group; and an aluminum complex group, and each group consists of 10 rabbits. According to the body weight of the rabbits, the rabbits are injected with the low dose solution of the extract of Typhaceae, the high dose solution of the extract of Typhaceae, the sodium derivative solution of the present invention, the aluminum complex solution of the present invention, the urokinase solution and the physiological saline solution. After four hours, each is injected with pentobarbital. Then fix the back of the rabbit and cut from the middle of the neck to separate a right carotic artery having a length of 1.5 cm. Use a small piece of cloth to cover the vicinity of the wound. Then use 2 electric probes to tilt the carotic artery lightly and apply 1.5 mA direct current stimulation. Use a semi-conductor probe type thermometer to fix the touching end of the carotic artery and measure the surface temperature of the artery continuously. Then record the occlusion time OT for a sudden drop of temperature and results are shown in table 8.

TABLE 8
The effect of the present invention on electrical stimulus resulting
carotic thrombosis of rabbits

| Group | Quantity | | OT (minute) |
|---|---|---|---|
| Physiological saline | — | | 38.9 ± 5.8 |
| solution | | | |
| Urokinase solution | 20000 | U | 48.8 ± 8.8* |
| Light dose | 1 | (mg/kg) | 58.1 ± 9.8** |
| Heavy dose | 3 | (mg/kg) | 68.2 ± 7.9** |
| Sodium derivative | 3 | (mg/kg) | 70.4 ± 6.5** |
| Aluminum complex | 3 | (mg/kg) | 64.4 ± 5.8** |

*P < 0.05,
**P < 0.01

Experiment 7: The Effect of the Present Invention on Bleeding and Coagulation Time of Mice.

A predetermined number of mice are randomly divided into a control group (i.e. same weight distilled water group); a low dose group; a heavy dose group; a sodium derivative group; an aluminum complex group and a Yunnan baiyao (a famous Traditional Chinese Medicine) group. Each is feed with a predetermined testing solution according to their group for two weeks. At 30-minute after each feeding, glass method is used to test the time of coagulation of the mice. The next day, bleeding time is obtained by cutting tail method. Results are shown in table 9.

TABLE 9
The effect of the present invention on bleeding and coagulation
time of mice.

| | Number | | | |
|---|---|---|---|---|
| | of | | | |
| | animal | Quantity | Coagulation time | Bleeding time |
| Group | (n) | (g/kg) | (second) | (second) |
| Control | 10 | | 250.16 ± 40.20 | 451.34 ± 178.40 |
| Yunnan | 10 | 0.6 | 413.15 ± 74.12*** | 584.90 ± 176.45 |
| baiyao | | | | |
| Light | 10 | 0.05 | 32.18 ± 30.56* | 212.12 ± 78.64*** |
| dose | | | | |
| Heavy | 10 | 0.15 | 390.68 ± 33.46** | 181.75 ± 60.52*** |
| dose | | | | |
| Sodium | 10 | 0.15 | 398.45 ± 23.33** | 176.32 ± 55.12*** |
| deriva- | | | | |
| tive | | | | |
| Alumi- | 10 | 0.15 | 366.23 ± 34.338** | 189.55 ± 34.89*** |
| num | | | | |
| com- | | | | |
| plex | | | | |

*P > 0.05,
**P < 0.05,
***P < 0.01

The present invention also includes the application of the extract of Typhaceae. The extract of Typhaceae of the present invention may be used independently as medicine and health supplement, or may be used together with western or Chinese health products or food, particularly used together with a type of Traditional Chinese medicine for increasing the metabolic rate and blood circulating as well as promoting bruise heeling or preventing blood vessels of the brain and heart, as a health supplement for specific purpose or a constituent of medicine.

Whether the extract of Typhaceae of the present invention is used independently or is used together with other western or Chinese health products or medicine, it will have the active effects of lowering blood lipid level, preventing arteriosclerosis, increasing coronary blood flow, increasing the tolerance ability of brain and cardiac muscle under anaerobic condition, preventing coagulating of blood platelet, preventing thrombosis and stop bleeding. Applications may be used for (I) preventing and treating heart and brain's vessels disease such as hyperlipemia, arteriosclerosis, coronary arterial disease, mycocardial infarction, cerebral thrombosis, cerebral vascular accident, and sequela of cerebral vascular accident; (II) preventing and treating different kinds of poor blood circulation induced problems such as chest pain, stomachache, physical injuries, puerperium pain and menstruation pain; (III) preventing and treating different kinds of bleeding such as hematemesis, bleeding, apistaxis, external bleeding, kidney malfunction, melana, endermic bleeding, internal bleeding and bleeding induced from wounding.

When the extract of Typhaceae of the present invention, or the medicine or health products or supplement containing the extract of Typhaceae of the present invention is used for promoting health or treatment purposes, the one skill in the art may utilizes one's knowledge, directly or with the addition of necessary constituents, to make the extract of the present invention into the form of capsule, tablet, injection velvet, granule, oral solution, syrup, ointment, alcoholic solution, drinks, juice, instant tea, and candy. When the extract of the present invention is manufactured as tablet, it will include determinate agent such as diluting solution such as starch, dextrin and lactose; wetting agent or agglutinant such as water, ethanol, starch solution, dextrin, gelatin solution, low substitution hydroxypropyl methylcellulose, polyethene pyrrolidone and polyethylene glycol; disintegration agent such as dry starch, bubbling disintegration agent and superficial active agent; and lubricant such as talcum powder, magnesium stearate, liquid wax, polyethylene glycol 6000 and 4000. When the extract of the present invention is manufactured in the form of capsule, it will include determinate agent including diluting agent such as starch, dextrine, lactose, magnesium oxide and magnesium carbonate; wetting agent or agglutinant such as water, ethanol, starch solution, dextrin, gelatin solution, low substitution hydroxypropyl methylcellulose, polyethene pyrrolidone and polyethylene glycol; disintegration agent such as dry starch, bubbling disintegration agent and superficial active agent; and hard or soft gelatin capsule. When the extract of the present invention is manufactured as a constituent for medical use in the form of injection velvet, it will include determinate agent having solublizing agent such as tween-80 and glycerol; mixing agent such as hydroxypropyl methylcellulose, polyethene pyrrolidone, and methylcellulose; antioxidant such as sodium sulfite, sodium pyrosulfite and sodium hyposulfite; osmoregulating agent such as sodium chloride and glucose; additional pain releaser such as benzyl alcohol and procaine hydrogen chloride. When the combination of the extract of Typha of the present invention is manufactured in the form of syrup or drinks, it will include a determinate agent including sucrose and taste modifying agent such as hydroxypropyl methylcellulose, polyethene pyrrolidone, and methylcellulose; and antiseptic such as ethyl paraben, methyl paraben, propylene glycol, benzoic acid and sorbitol.

The present invention is to provide an extract of a Typhae Pollen and its manufacture and application in medicine and health science. The present invention is described clearly and explicitly in the following embodiments.

The first example of a first preferred embodiment is a process of preparing flavonoids of Typhaceae from crude materials, wherein the process of preparing flavonoids of Typhaceae from crude materials comprises the steps of:

(1a) providing 1 kg crude Typhaceae and placing the crude Typhaceae in an container;

(1b) adding 7 kg 70% ethanol and mixing thoroughly;

(1c) heating under water bath and reflux for 2 hours;

(1d) filtering immediately after step (1c) to obtain a first extract solution and separating out a residue Typha

(1e) using 5 kg 70% ethanol to reflux for 1 hour and filtering immediately to obtain a second extract solution; and

(1f) mixing the first extract solution and the second extract solution to form a filtrate solution.

According to the first example of the first preferred embodiment, the filtrate solution is recollected to form a condense patch having a weight between 1.1-1.2 by using a rotatory evaporator to reduce pressure. Using a 1:10 ratio regulation, 10% ethanol solution is added and chitosan as fining agent having 1% of total volume of the condense patch is added to form a preliminary extract solution. Then the preliminary extract solution is settled under room conditions and filtered by centrifugation to form a centrifuged solution. Adsorption of the centrifuged solution is carried out by using a predetermined prepared HZ-802 macroporous resin (manufactured by Shanghai Hua Zeng Technology Trading Company) located in a stationary bed. After the centrifuged solution is treated by adsorption and then filtration, 8-10 liter de-ionizing solution is used to wash the stationary bed until it is clear. 10-12 liter 30% ethanol is then used to wash the stationary bed until the color goes pale. Lastly, a dried sample is obtained washing by 8-10 liter 80% ethanol to collect a 80% washing solution which forms a second condensed patch after reducing pressure and recollected by rotatory evaporator, and placing the second condensed patch in vacuum dryer to vacuum drying. The dried sample is broke and the extract of Typha is obtained, namely a resulting extract. After analysis, a percentage of flavonoids is 62.5%.

The second example of a first preferred embodiment is a process of preparing flavonoids of Typhaceae from crude materials, wherein the process of preparing flavonoids of Typhaceae from crude materials comprises the steps of:

(2a) providing 1 kg crude Typhaceae and placing the crude Typhaceae in an container;

(2b) adding 7 kg 70% ethanol and mixing thoroughly;

(2c) heating under water bath and reflux for 2 hours;

(2d) filtering immediately after step (2c) to obtain a first extract solution and separating out a residue;

(2e) using 5 kg 70% ethanol to reflux for 1 hour and filtering immediately to obtain a second extract solution; and

(2f) mixing the first extract solution and the second extract solution to form a filtrate solution.

According to the second example of the first preferred embodiment, the filtrate solution is recollected to form a condense patch having a weight between 3L (percentage weight is 1.05) by using a rotatory evaporator to reduce pressure. Then 500 ml aqueous solution of saturated lead acetate is added and stirred thoroughly to form a mixture solution. The mixture solution is settled and a sediment is obtained by centrifugation. The sediment is then mixed with 2.5L 95% ethanol and double decomposition is carried out through H₂S, which is then centrifuged to remove lead sulfate sediment and a filtrate containing ethanol is recollected by filtration. The filtrate is dried by vacuum drying and broken to obtain a 143 g extract of Typha, namely a resulting extract. After analysis, the percentage of flavonoids is 54.2%.

The third example of the first preferred embodiment of the present invention is a process of making a highly concentrated extract of Typhaceae which comprises the steps of:

(3a) obtaining a 5 g predetermined starting materials which is the resulting extract from the first or second example;

(3b) dissolve the starting materials with a predetermined volume of methanol to form a starting solution;

(3c) using 500 g Sephadex-LH20 as solvent for column chromatography of the starting solution;

(3d) washing with methanol and collecting major color column separately;

(3e) recollecting solvent until drying up; and

(3f) obtaining a 1.25 g isorhamnetin-3-O-(2^{G}-α-L-rham)-rutinoside and a 0.98 g isorhamnetin-3-O-neohesperidoside separately.

After analysis, the percentage of the isorhamnetin-3-O-(2^{G}-α-L-rham)-rutinoside is 96.4% and the percentage of the isorhamnetin-3-O-neohesperidoside is 98.5%.

The forth example of the first preferred embodiment of the present invention is a process of preparing a sodium derivative of the present invention which comprises the steps of:

(4a) obtaining a 100 g predetermined starting materials which is the resulting extract from the first or second example;

(4b) dissolving the starting materials in 500 ml water to form a starting solution;

(4c) adding a 0.5N sodium bicarbonate solution to the starting solution until the pH is 8 and mixing the sodium bicarbonate solution and the starting solution thoroughly;

(4d) removing insoluble impurities by centrifugation (8000 rps) and obtaining a preliminary extract solution; and

(4e) obtaining a 89 g sodium derivative of the extract of Typhaceae by reducing pressure, condensation and vacuum drying of the preliminary extract solution.

After analysis, the percentage of the sodium derivative is between 58% and 65.5%.

The fifth example of the first preferred embodiment of the present invention is a process of preparing a aluminum complex of the present invention which comprises the steps of:

(5a) obtaining a 100 g predetermined starting materials which is the resulting extract from the first or second example;

(5b) dissolving the starting materials in 500 ml water to form a starting solution;

(5c) adding a 100 ml 10% aluminum trichloride solution to the starting solution;

(5d) mixing the aluminum trichloride solution and the starting solution thoroughly and heating at 90° C. for 1 hour to form a mixture solution;

(5e) removing insoluble substances of the mixture solution by centrifugation (8000 rps) after cooling to form a preliminary extract solution;

(5f) obtaining a 95 g aluminum complex of the extract of Typha by reducing pressure, condensation and vacuum drying of the preliminary extract solution.

After analysis, the percentage of the aluminum complex is between 54.3% and 62.5%.

The sixth example of the first preferred embodiment of the present invention is a process of making an extract of Typhaceae in the form of tablet which comprises the steps of

(6a) providing a 100 g extract of Typhaceae and a 100 g starch; and

(6b) mixing the extract and the starch to form a mixture and filling the mixture into a gelatin capsule.

The seventh example of the first preferred embodiment of the present invention is a process of making an extract of Typhaceae as one of the constituents of a product which comprises the steps of:

(7a) providing starting materials which are a 50 g extract of Typhaceae, a 50 g extract of faece of Trogopterus Xanthipes Milne having process of water extraction and alcoholic condensation, a 20 g camphol and a 80 g starch; and

(7b) mixing the starting materials and filling the mixed starting materials into a gelatin capsule.

One skilled in the art will understand that the embodiment of the present invention described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A flavonoids extract obtained from a traditional Chinese medicine Typhae Pollen, comprising flavonoids constituents including a kaempferol, a kaempferol-3-O-neohesperidoside, a kaempferol-3-O-(2^{G}-α-L-rham)-rutinoside, a kaempferol-3-rutino-7-rhamnoside, a quercetin, a quercetin-3-O-neohesperidoside, a quercetin-3-O-(2^{G}-α-L-rham)-rutinoside, a quercetin-3-rutino-7-rhamnoside, an isorhamnetin, an isorhamnetin-3-O-neohesperidoside, an isorhamnetin-3-O-(2^{G}-α-L-rham)-rutinoside, and an isorhamnetin-3-rutino-7-rhamnoside.

2. The flavonoids extract, as recited in claim 1, wherein said Typhae Pollen having a plant body is a common name of a family Typhaceae and is at least one of the portions of said plant body selecting from a whole plant body, a pollen, a spica, a stem, a leave, a fruit, a root and a rootstalk.

3. The flavonoids extract, as recited in claim 1, wherein said Typhae Pollen having a plant body is a common name of a family Typhaceae and is originated from a mature pollen of the plant body.

4. The flavonoids extract, as recited in claim 1, wherein said Typhae Pollen is capable of having the forms selecting from the group consisting of a crude form which is the crude Typhae Pollen and a non-crude form which is treated, wherein said non-crude form is capable of selecting from the group consisting of fried Typhae Pollen, brunt Typhae Pollen, alcoholic Typhae Pollen and vinegar Typhae Pollen.

5. The flavonoids extract, as recited in claim 1, wherein a total percentage composition of said plurality of constituents is in the range between 5% and 100% by weight.

6. The flavonoids extract, as recited in claim 5, wherein a percentage composition of said isorhamnetin-3-O-(2^{G}-α-L-rham)-rutinoside and said isorhamnetin-3-O-neohesperidoside is a range of 20% and 100% by weight.

7. The flavonoids extract, as recited in claim 1, wherein said flavonoids constituents are in a degraded form produced by a process of degradation carried out under a predetermined condition selecting from the group of heating condition, acidic condition, alkaline condition and enzyme condition.

8. The flavonoids extract, as recited in claim 1, wherein said flavonoids constituents are in a metal derivative form produced by a reaction with a predetermined metal salt selecting from the group consisting of a sodium salt and a potassium salt.

9. The flavonoids extract, as recited in claim 1, wherein said flavonoids constituents are in a metal complex form produced by a reaction with a predetermined metal ion selecting from the group consisting of zinc ion, magnesium ion, chromium ion, iron ion, aluminum ion, copper ion, calcium ion, cobalt ion, barium ion, strontium ion, and zirconium ion.

10. The flavonoids extract, as recited in claim 1, wherein said flavonoids extract is produced from a predetermined process of extraction selecting from the group consisting of a solvent extraction process; a macroporous resin adsorption process; a lead salt precipitation process; a supercritical CO₂ extraction process; a column chromatography process and a liquid-liquid reflux extraction process.

11. The flavonoids extract, as recited in claim 10, wherein said solvent extraction process comprises the steps of:
(a) providing a predetermined starting extract materials in water;
(b) removing oil soluble impurities by a low polarity agent selected from the group consisting of ether, alkane, and ester solvent; and
(c) obtaining a resulting flavonoids extract by applying at least one predetermined polar solvent selecting from the group consisting of butanol, isopropanol and chloroform.

12. The flavonoids extract, as recited in claim 10, wherein said macroporous resin adsorption process comprises the steps of:
(a) providing a predetermined resin selected from the group consisting of non-polar resin, low polarity resin, medium polarity resin, weak basicity resin and weak acidity resin; and
(b) using a predetermined extracting agent selected from the group consisting of water, ethanol in water, methanol in water, and propanone in water for extracting a resulting flavonoids extract.

13. The flavonoids extract, as recited in claim 10, wherein said lead salt precipitation process comprises the steps of
(a) providing a predetermined lead salt agent selecting from the group consisting of lead acetate and basic lead acetate; and
(b) using a predetermined demineralizing agent selecting from the group consisting of H2S, phosphate and sulfate for extracting a resulting flavonoids extract.

14. The flavonoids extract, as recited in claim 10, wherein said column chromatography process comprises the steps of
(a) providing predetermined starting extract materials;
(b) providing a stationary bed having materials selected from the group consisting of silica gel, polyamide, aluminum oxide, polysaccharide, C-8, C-18, active charcoal, and cellulase; and
(c) using a mixing eluting agent selecting from at least two of the group consisting of water, methanol, ethanol and propanone, chloroform and ethyl acetate to obtain a resulting flavonoids extract.

15. The flavonoids extract, as recited in claim 14, wherein said starting extract materials is a preliminary purifying substance obtained from a purification process, wherein said purification process comprises a step of purifying a flavonoids extract prepared by a predetermined preliminary extraction process selecting from the group consisting of solvent extraction process, macroporous resin adsorption process, lead salt precipitation and supercritical CO₂ extraction process.

16. The flavonoids extract, as recited in claim 10, wherein said liquid-liquid reflux extraction process comprises the steps of
(a) providing a predetermined starting extract materials in water;
(b) removing oil soluble impurities by a low polarity agent selected from the group consisting of ether, alkane, and ester solvent; and
(c) obtaining a resulting flavonoids extract by applying at least one predetermined polar solvent selected from the group consisting of butanol, isopropanol and chloroform.

17. The flavonoids extract, as recited in claim 16, wherein said starting extract materials is a preliminary purifying substance obtained from a purification process, wherein said purification process comprises a step of purifying a flavonoids extract prepared by a predetermined preliminary extraction process selecting from the group consisting of solvent extraction process, macroporous resin adsorption process, lead salt precipitation and supercritical CO₂ extraction process.

18. A method of preparing a flavonoids extract including a kaempferol, a kaempferol-3-O-neohesperidoside, a kaempferol-3-O-(2^{G}-α-L-rham)-rutinoside, a kaempferol-3-rutino-7-rhamnoside, a quercetin; a quercetin-3-O-neohesperidoside, a quercetin-3-O-(2^{G}-α-L-rham)-rutinoside, a quercetin-3-rutino-7-rhamnoside, an isorhamnetin, an isorhamnetin-3-O-neohesperidoside, an isorhamnetin-3-O-(2^{G}-α-L-rham)-rutinoside, and an isorhamnetin-3-rutino-7-rhamnoside, wherein said method comprises an extraction process, a filtration process, a condensation process, and a drying process.

19. The method, as recited in claim 18, wherein said extraction process comprising the steps of:
providing a predetermined solvent selecting from the group consisting of water, acetate solvent, ketone solvent, and ester solvent, wherein said predetermined solvent is capable of reacting with a predetermined substance selecting from the group consisting of acid and alkali to form an acidic solvent and an alkaline solvent respectively such that said acidic solvent and said alkaline solvent are capable of used to substitute said predetermined solvent; and
obtaining said flavonoids extract by a method of solvent extraction selecting from the group consisting of refluxation, diffusion, ultraextraction, microextraction and pressure extraction.

20. The method, as recited in claim 19, wherein said predetermined solvent is 70% ethanol.

21. The method, as recited in claim 18, wherein said filtration process is selected from the process consisting of centrifugation, extract filtration, pressure filtration, ultrafiltration.

22. The method, as recited in claim 21, wherein said filtration process comprises a step of providing a fining agent selecting from the group consisting of gelatin, active charcoal, infusorial earth, china clay; resins, polyethylene glycol, polyethene triol, chitsoan, and natural fining agent.

23. The method, as recited in claim 18, wherein said condensation process is a process selecting from the group consisting of film evaporation, rotatory evaporation and heating under a normal pressure condition.

24. The method, as recited in claim 18, wherein said drying process is a process selected from the group consisting of vacuum drying, spray drying and freeze drying.

25. A method for treating patients with blood diseases comprising administering said flavonoides extract according to claim 1 to patients with said blood diseases.

26. The method, as recited in claim 25, wherein said blood diseases are diseases of blood vessel in brain and heart

27. The method, as recited in claim 25, wherein said blood diseases of said patients are poor blood circulation induced diseases selected from the group consisting of chest pain, stomachache, physical injuries, puerperium pain and menstruation.

28. The method, as recited in claim 25, wherein said blood diseases are diseases involving bleeding.

29. A method for preventing users with blood diseases comprising administering said flavonoides extract according to claim 1 to said users to prevent said users to have said blood diseases.

30. The method, as recited in claim 29, wherein said blood diseases are diseases of blood vessel in brain and heart

31. The method, as recited in claim 29, wherein said blood diseases of said patients are poor blood circulation induced diseases selected from the group consisting of chest pain, stomachache, physical injuries, puerperium pain and menstruation.

32. The method, as recited in claim 29, wherein said blood diseases are diseases involving bleeding.

33. An extract of Typhae Pollen wherein one of said extract components is flavonol glycosides and said active components of said flavonol glycosides are a combination of the group selected from structural formulae (A), (B) and (C), wherein said structural formula (A) consists of chemical components (1), (2), (3) and (4); said structural formula (B) consists of chemical components (5), (6), (7) and (8); and said structural formula (C) consists of chemical components (9), (10), (11), (12) and (13);
wherein said structural formula (A) is: 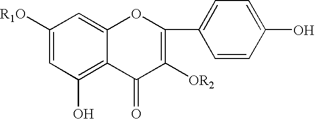
where said chemical component (1) is R₁=R₂=H, said chemical component (2) is R₁=H, R₂=neohesperidoside, said chemical component (3) is R₁=H, R₂=(2^{G}-rham)-rutinoside, and said chemical component (4) is R₁=rhamnoside, R₂=rutinoside;
wherein said structural formula (B) is: 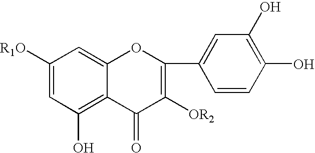
where said chemical component (5) is R₁=R₂=H, said chemical component (6) is R₁=H, R₂=neohesperidoside, said chemical component (7) is R₁=H, R₂=(2^{G}-rham)-rutinoside, and said chemical component (8) is R₁=rhamnoside, R₂=rutinoside;
wherein said structural formula (C) is: 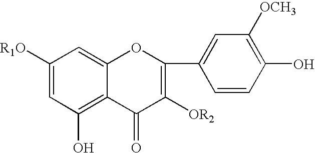
where said chemical component (9) is R₁=R₂=H, said chemical component (10) is R₁=H, R₂=neohesperidoside, said chemical component (11) is R₁=R₂=rutinoside, said chemical component (12) is R₁=H, R₂=(2G-rham)-rutinoside, and said chemical component (13) is R₁=rhamnoside, R₂=rutinoside.

34. The extract of Typhae Pollen, as recited in claim 33, wherein said chemical components (1), (2), (3), (4), (5), (6), (7), (8), (9), (10), (11), (12) and (13) are kaempferol, kaempferol-3-O-neohesperidoside, kaempferol-3-O-(2^{G}-α-L-rham)-rutinoside, kaempferol-3-rutino-7-rhamnoside, quercetin, quercetin-3-O-neohesperidoside, quercetin-3-O-(2^{G}-α-L-rham)-rutinoside, quercetin-3-rutino-7-rhamnoside, isorhamnetin, isorhamnetin-3-O-neohesperidoside, isorhamnetin-3-O-(2^{G}-α-L-rham)-rutinoside and isorhamnetin-3-rutino-7-rhamnoside, respectively.
